(19) United States
(12) Patent Application Publication
(10) Pub. No.: US 2026/0026836 A1
(43) Pub. Date: Jan. 29, 2026

(54) ULTRASONIC TREATMENT TOOL

(71) Applicant: OLYMPUS MEDICAL SYSTEMS CORP., Tokyo (JP)

(72) Inventors: Tsubasa NIIYAMA, Tokyo (JP); Marina ASAHINA, Sagamihara-shi (JP); Keisuke NAGAO, Tokyo (JP); Kaito SATO, Tokyo (JP)

(21) Appl. No.: 19/038,400

(22) Filed: Jan. 27, 2025

Related U.S. Application Data
(63) Continuation of application No. PCT/JP2024/026713, filed on Jul. 25, 2024.

(51) Int. Cl. A61B 17/32; A61B 17/00; A61B 90/00

(52) U.S. Cl. CPC A61B 17/320092; A61B 90/03; A61B 2017/00477; A61B 2017/00836; A61B 2017/00862; A61B 2017/00929; A61B 2017/00938; A61B 2017/320075; A61B 2017/320094; A61B 2090/034

(57) ABSTRACT

An ultrasonic treatment tool includes: an ultrasonic blade configured to supply ultrasonic vibration and high frequency current to a living tissue; a jaw configured to open and close with respect to the ultrasonic blade; a holder that is supported by the jaw, and that is made of a first material having an electrical insulation property; an abutting portion that is arranged in the holder, and that is made of a first resin material, the abutting portion being configured to abut on the ultrasonic blade when the jaw is closed with respect to the ultrasonic blade; and a first pin that is provided in the jaw to support the holder in a swingable manner.

### CROSS REFERENCES TO RELATED APPLICATIONS

This application is a continuation of International Application No. PCT/JP2024/026713, filed on Jul. 25, 2024, the entire contents of which are incorporated herein by reference.

### BACKGROUND

### 1. Technical Field

The present disclosure relates to an ultrasonic treatment tool.

### 2. Related Art

In the related art, an ultrasonic treatment tool has been known that supplies ultrasonic vibrations and high frequency current as treatment energy to a part to be a target of treatment a living tissue (hereinafter, referred to as “treatment target”), to treat the treatment target (for example, JP-T-2019-509775).

The ultrasonic treatment tool described in JP-T-2019-509775 includes an ultrasonic blade, a jaw, and an abutting portion described below. The ultrasonic blade supplies ultrasonic vibrations and high frequency current to a treatment target. The jaw opens and closes with respect to the ultrasonic blade. Moreover, in the jaw, an electrode to supply the high frequency current to a treatment target is arranged. The abutting portion is made of a resin material, and is arranged in the jaw. The abutting portion abuts on the ultrasonic blade when the jaw closes with respect to the ultrasonic blade.

### SUMMARY

In some embodiments, an ultrasonic treatment tool includes: an ultrasonic blade configured to supply ultrasonic vibration and high frequency current to a living tissue; a jaw configured to open and close with respect to the ultrasonic blade; a holder that is supported by the jaw, and that is made of a first material having an electrical insulation property; an abutting portion that is arranged in the holder, and that is made of a first resin material, the abutting portion being configured to abut on the ultrasonic blade when the jaw is closed with respect to the ultrasonic blade; and a first pin that is provided in the jaw to support the holder in a swingable manner. The holder includes an opposing surface that faces the ultrasonic blade, and an electrode surface that is positioned in a direction away from the ultrasonic blade with respect to the opposing surface and on which an electrode is arranged, the electrode being electrically connected to the jaw through the first pin to supply the high frequency current.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an ultrasonic treatment tool according to a first embodiment;

FIG. 2 is a diagram explaining a configuration of a distal end portion in the ultrasonic treatment tool;

FIG. 3 is a diagram explaining a configuration of a distal end portion in the ultrasonic treatment tool;

FIG. 4 is a configuration diagram of an ultrasonic blade;

FIG. 5 is a diagram illustrating a partial cross-section on a proximal end side of the ultrasonic blade;

FIG. 6 is a diagram explaining a configuration of a jaw;

FIG. 7 is a diagram explaining a configuration of a holder;

FIG. 8 is a partial enlarged view in which an electrode surface in FIG. 3 is enlarged;

FIG. 9 is a diagram illustrating an ultrasonic treatment tool according to a second embodiment;

FIG. 10 is a partial enlarged view in which an electrode surface is enlarged; and

FIG. 11 is a diagram illustrating a top surface and a side surface of a jaw of a treatment tool according to a third embodiment.

### DETAILED DESCRIPTION

Hereinafter, forms to implement the disclosure (hereinafter, embodiments) will be explained with reference to the drawings. The embodiments explained below are not intended to limit the disclosure. Furthermore, in descriptions of the drawings, like reference symbols are assigned to like parts.

### First Embodiment

### Schematic Configuration of Treatment System

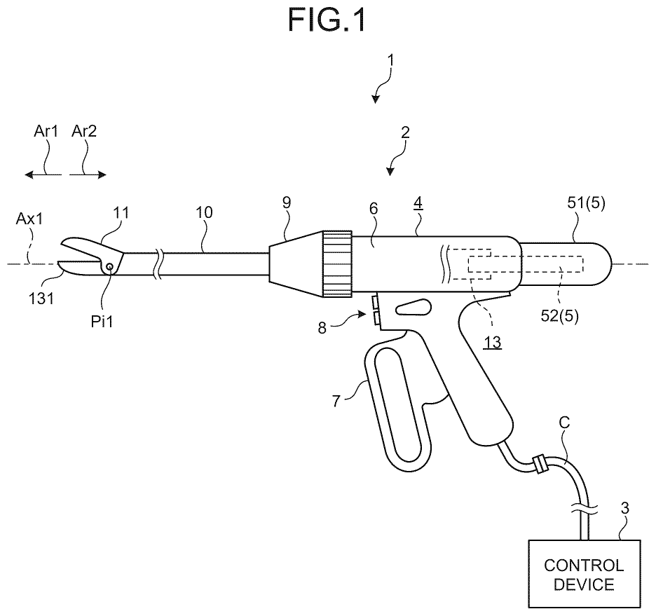

FIG. 1 is a diagram illustrating an ultrasonic treatment tool according to a first embodiment. A treatment system 1 applies a treatment energy to a site to be a target of treatment in a living tissue (hereinafter, denoted as treatment target), to thereby treat the treatment target. The treatment energy in the first embodiment is an ultrasonic energy and a high frequency energy. Moreover, the treatment possible to be performed by the treatment system 1 according to the first embodiment includes treatments, such as coagulation (sealing) of a treatment target and incision of a treatment target. Furthermore, coagulation and incision may be performed at the same time. This treatment system 1 includes a treatment tool 2 and a control device 3 as illustrated in FIG. 1.

### Configuration of Treatment Tool

In the following, one side along a center axis Ax1 (FIG. 1) of an outer pipe 10 is referred to as distal end side Ar1, and the other side is referred to as proximal end side Ar2. Moreover, “width direction” described in the following is a direction perpendicular to the center axis Ax1 and an opening closing direction of a jaw 11 with respect to a treating portion 131, and signifies a direction perpendicular to the sheet plane of FIG. 1.

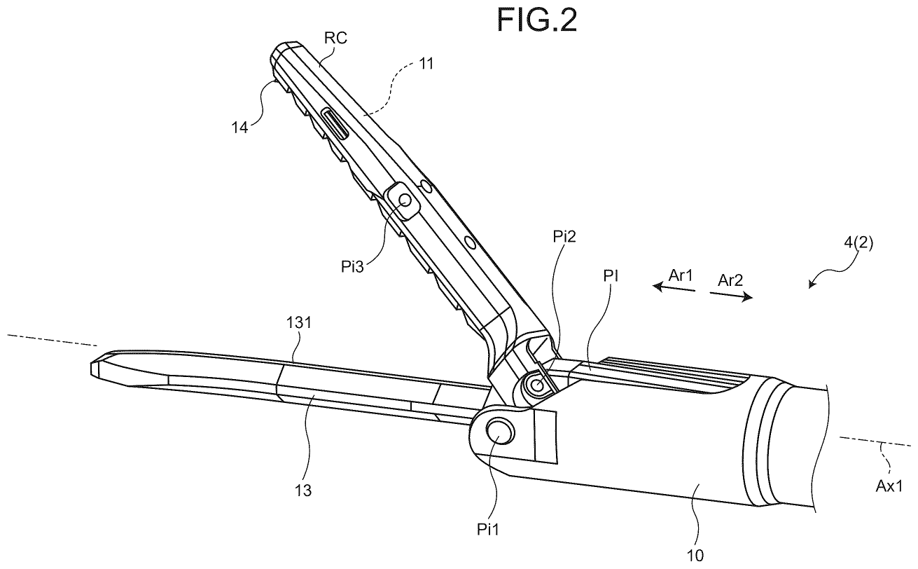

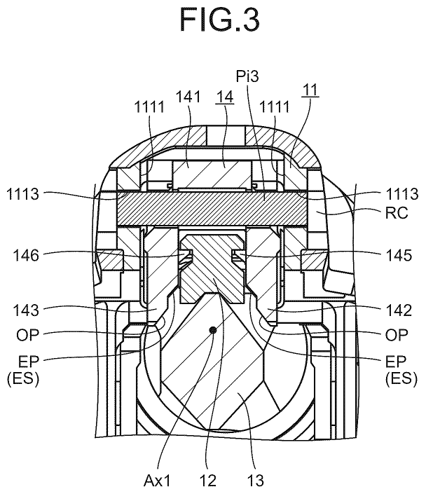

FIG. 2 and FIG. 3 are diagrams explaining a configuration of a distal end portion of the treatment tool 2. Specifically, FIG. 2 is a perspective view illustrating the distal end portion of the treatment tool 2. FIG. 3 is a cross-section of the distal end portion of the treatment tool 2 cut along a plane perpendicular to the center axis Ax1.

The treatment tool 2 is the ultrasonic treatment tool. The treatment tool 2 applies an ultrasonic energy and a high frequency energy to a treatment target, to thereby treat the treatment target. The treatment tool 2 includes a handpiece 4 and an ultrasonic transducer 5 as illustrated in FIG. 1.

The handpiece 4 includes, as illustrated in FIG. 1 to FIG. 3, a fixed handle 6 (FIG. 1), an operation handle 7 (FIG. 1), a switch 8 (FIG. 1), a rotating knob (FIG. 1), the outer pipe 10 (FIG. 1, FIG. 2), the jaw 11, an abutting portion 12 (FIG. 3), an ultrasonic blade 13, and a holder 14 (FIG. 2, FIG. 3).

The fixed handle 6 is a portion that supports the entire treatment tool 2, and that is held by an operator, such as a surgeon (user).

The operation handle 7 is movably attached to the fixed handle 6, and receives an opening closing operation of the jaw 11 by the operator, such as a surgeon.

The switch 8 is arranged on an exterior of the fixed handle 6 in an exposed state, and receives a treatment operation by the operator, such as a surgeon.

A rotating knob 9 has a substantially cylindrical shape coaxial with the center axis Ax1, and is arranged at the distal end side Ar1 of the fixed handle 6. The rotating knob 9 receives a rotating operation by the operator, such as a surgeon. By the rotating operation, the rotating knob 9 rotates about the center axis Ax1 with respect to the fixed handle 6. Moreover, by the rotation of the rotating knob 9, the outer pipe 10, the jaw 11, the holder 14, the abutting portion 12, and the ultrasonic blade 13 rotate about the center axis Ax1.

The outer pipe 10 has a tubular shape, and corresponds to a pipe. In the first embodiment, the outer pipe 10 is a cylindrical pipe made of an electrically conductive material, such as metal.

In this outer pipe 10, a first pin Pi1 (FIG. 1, FIG. 2) that has a cylindrical shape extending in the direction perpendicular to the sheet plane of FIG. 1, and that engages with the jaw 11 and axially supports the jaw 11 in a rotatable manner is fixed at an end portion on the distal end side Ar1. In the first embodiment, the first pin Pi1 is made of an electrically conductive material, such as metal.

An outer circumferential surface of the outer pipe 10 is covered with an electrically insulating outer tube (not illustrated). Moreover, inside the outer pipe 10, a tubular-shaped inner pipe PI (FIG. 2) that moves back and forth along a longitudinal direction of the outer pipe 10 in response to the opening closing operation to the operation handle 7 by operator, such as a surgeon, is inserted. At an end portion on the distal end side Ar1 of the inner pipe PI, a second pin Pi2 (FIG. 2) that has a cylindrical shape extending in the direction perpendicular to the sheet plane of FIG. 1, and that engages with the jaw 11 is fixed. In the first embodiment, the second pin Pi2 is arranged on an upper side with respect to the first pin Pi1 in FIG. 2 (a side on which a jaw main body 111 is arranged with respect to the treating portion 131).

The jaw 11 is connected to the outer pipe 10 by the first pin Pi1. Moreover, the jaw 11 is connected to the inner pipe PI by the second pin Pi2. The jaw 11 rotates around the first pin Pi1 with respect to the outer pipe 10 in conjunction with the back and forth movement of the inner pipe PI according to the opening closing operation to the operation handle 7 by the operator, such as surgeon. Thus, the jaw 11 opens and closes with respect to the treating portion 131, which is the end portion on the distal end side in the ultrasonic blade 13, and is enabled to hold the treatment target between itself and the treating portion 131.

The treatment tool 2 may be configured as a push-to-close type, or a pull-to-close type.

The push-to-close type has a following configuration.

The jaw 11 rotates in a direction approaching the treating portion 131 around the first pin Pi1 in conjunction with movement of the inner pipe PI to the distal end side Ar1. That is, the jaw 11 closes with respect to the treating portion 131. Moreover, the jaw 11 rotates in a direction away from the treating portion 131 around the first pin Pi1 in conjunction with movement of the inner pipe PI to the proximal end side Ar2. That is, the jaw 11 opens with respect to the treating portion 131.

The pull-to-close type has a following configuration.

The jaw 11 rotates in the direction approaching the treating portion 131 around the first pin Pi1 in conjunction with movement of the inner pipe PI to the proximal end side Ar2. That is, the jaw 11 closes with respect to the treating portion 131. Moreover, the jaw 11 rotates in a direction away from the treating portion 131 around the first pin Pi1 in conjunction with movement of the inner pipe PI to the distal end side Ar1. That is, the jaw 11 opens with respect to the treating portion 131.

The jaw 11 may be configured such that the outer pipe 10 moves back and forth according to the opening closing operation to the operation handle 7. In this case, the jaw 11 rotates around the second pin Pi2 in conjunction with the back and forth movement of the outer pipe 10, to open and close with respect to the treating portion 131.

A detailed configuration of the jaw 11 will be explained in “About Configuration of Jaw” described later.

The holder 14 extends in a direction perpendicular to the sheet plane of FIG. 3, and is supported by a third pin Pi3 (FIG. 2) fixed to the jaw 11, swingably about a center axis of the third pin Pi3, and rotatably around the jaw 11. By arranging the holder 14 swingable about the center axis of the third pin Pi3, a position at which the strongest force is applied to the treatment target when the treatment target is held between the jaw 11 and the treating portion 131 is positioned approximately at the center of the jaw 11 in a longitudinal direction, not on the proximal end side Ar2 of the jaw 11. That is, to the treatment target held between the jaw 11 and the treating portion 131, the force is applied substantially uniformly. In the first embodiment, the third pin Pi3 is made of an electrically conductive material such as metal and has a cylindrical shape.

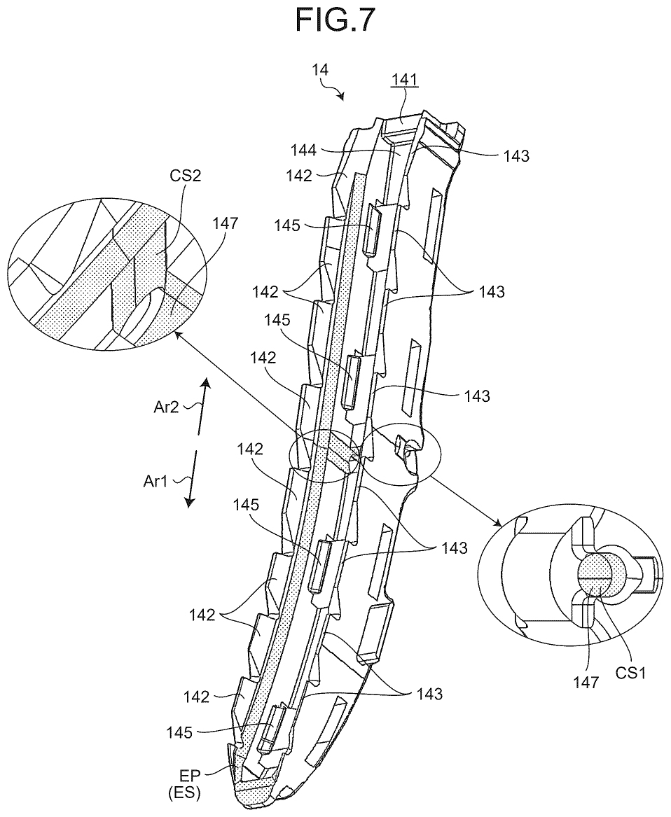

Moreover, in the holder 14, an electrode EP and first and second conductive surfaces CS1 and CS2 are arranged (refer to FIG. 7).

A detailed configuration of the holder 14 will be explained in “About Configuration of Holder” described later. Furthermore, configurations of the electrode EP and the first and the second conductive surfaces CS1 and CS2 will be explained in “About Configurations of Electrode and First and Second Conductive Surfaces” described later.

The abutting portion 12 is made of a first resin material having an electrical insulation property and a biocompatibility. The first resin material is, for example, polytetrafluoroethylene (PTFE). The abutting portion 12 has a substantially cuboid shape that extends along the longitudinal direction of the jaw 11 and the holder 14. The abutting portion 12 is fixed to a surface on a side closer to the treating portion 131 in a holder main body 141 as illustrated in FIG. 2 and FIG. 3, and abuts on the treating portion 131 when the jaw 11 is closed with respect to the treating portion 131. This abutting portion 12 has a function of preventing breakage caused when the treating portion 131 that is making ultrasonic vibrations collides with the jaw 11 after incision of the treatment target utilizing ultrasonic vibrations is completed.

The ultrasonic blade 13 is made of an electrically conductive material, and has a long shape extending along the center axis Ax1. Moreover, the ultrasonic blade 13 is inserted in the inner pipe PI in a state in which the treating portion 131 protrudes outside. At this time, the end portion on the proximal end side Ar2 of the ultrasonic blade 13 is mechanically connected to an ultrasonic vibrator 52 constituting the ultrasonic transducer 5. The ultrasonic blade 13 transmits ultrasonic vibrations generated by the ultrasonic transducer 5 from the end portion on the proximal end side Ar2 to the treating portion 131. The ultrasonic vibrations are vertical vibrations vibrating in the direction along the center axis Ax1. Furthermore, in the ultrasonic blade 13, an outer circumferential surface except the treating portion 131 is covered with the electrically insulating inner tube.

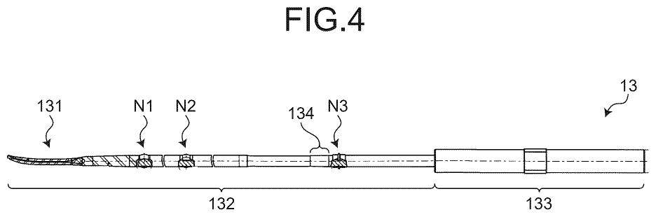

FIG. 4 is a configuration diagram of the ultrasonic blade. As illustrated in FIG. 4, the ultrasonic blade 13 is positioned on the distal end side, and includes a distal end portion 132 including the treating portion 131, and a proximal end portion 133 positioned on the proximal end side. The thickness of the distal end portion 132 is, for example, 3 mm or less, and the thickness of the proximal end portion 133 is, for example, 3 mm or more. Moreover, for a first node N1, a second node N2, and a third node N3 of the ultrasonic vibrations of the ultrasonic blade 13, a thin gain portion 134 is arranged between the node N2 and the node N3. By arranging the gain portion 134 on the proximal end side relative to the node N2, it is possible to increase the rigidity of the distal end portion 132 compared to a configuration in which a gain portion is positioned on the distal end side relative to the node N2, and to reduce horizontal vibrations at the distal end of the ultrasonic blade 13.

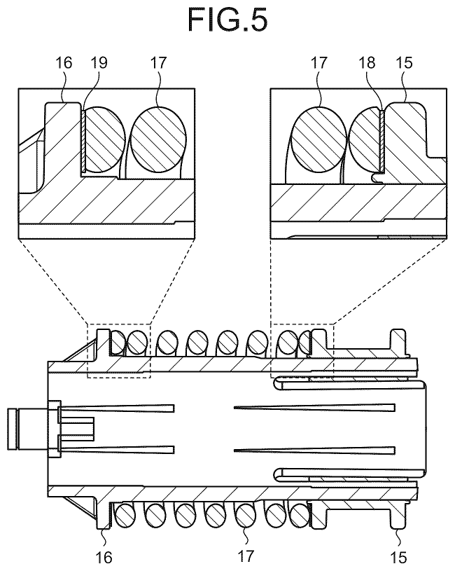

FIG. 5 is a diagram illustrating a partial cross-section of the ultrasonic blade on the proximal end side. On the proximal end side of the ultrasonic blade 13, the inner pipe PI is arranged on an outer circumference of the ultrasonic blade 13, and a slider 15, a slider receiver 16, and a coil spring 17 are arranged on an outer circumference of the inner pipe PI. The slider 15 moves back and forth according to the opening closing operation to the operation handle 7. The slider receiver 16 receives a pushing force from the slider 15 through the coil spring 17 arranged between the slider receiver 16 and the slider 15, and moves back and forth in conjunction with the inner pipe PI. Moreover, as illustrated in an enlarged view of a broken line portion in FIG. 5, a slider-side shim 18 is arranged on the proximal end side of the coil spring 17, and a slider-receiver-side shim 19 is arranged on the distal end side of the coil spring. The slider-side shim 18 and the slider-receiver-side shim 19 have a disc shape with a hole in the center, and are, for example, made of metal. By arranging the slider-side shim 18 and the slider-receiver-side shim 19, sterilization can be improved.

The ultrasonic transducer 5 includes a transducer (TD) case 51 and the ultrasonic vibrator 52 as illustrated in FIG. 1.

The TD case 51 supports and connects the ultrasonic vibrator 52 to the fixed handle 6.

The ultrasonic vibrator 52 generates ultrasonic vibrations under control of the control device 3. In the first embodiment, the ultrasonic vibrator 52 is constituted of a bolt-clamped Langevin-type transducer (BLT).

### Configuration of Control Device

The control device 3 comprehensively controls operation of the treatment tool 2 through an electrical cable C (FIG. 1). Specifically, the control device 3 detects a treatment operation to the switch 8 made by the operator, such as surgeon, through the electrical cable C. When the treatment operation is detected, the control device 3 applies a treatment energy to the treatment target held between the jaw 11 and the treating portion 131 through the electrical cable C. That is, the control device 3 treats the treatment target.

For example, when the ultrasonic energy is applied to a treatment target, the control device 3 supplies a driving power to the ultrasonic vibrator 52 through the electrical cable C. The ultrasonic vibrator 52 thereby generates vertical vibrations (ultrasonic vibrations) that vibrate in the direction along the center axis Ax1. Moreover, the treating portion 131 vibrates at a desired amplitude with the vertical vibrations. To the treatment target held between the jaw 11 and the treating portion 131, the ultrasonic vibrations are transmitted from the treating portion 131. The ultrasonic energy is applied to the treatment target from the treating portion 131.

Moreover, for example, when the high frequency energy is applied to a treatment target, the control device 3 supplies a high frequency power to a portion between the electrode EP arranged at the holder 14 and the ultrasonic blade 13 through the electrical cable C. When the high frequency power is supplied to the electrode EP and the ultrasonic blade 13, high frequency current is supplied to a living tissue, which is the treatment target, held between the jaw 11 and the treating portion 131. In other words, the high frequency energy is applied to the treatment target.

### About Configuration of Jaw

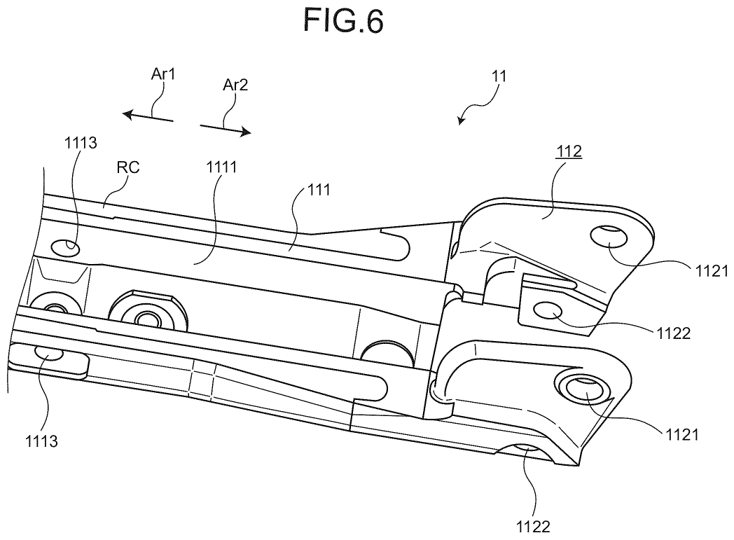

FIG. 6 is a diagram explaining a configuration of the jaw 11. Specifically, FIG. 6 is a perspective view as viewed from the treating portion 131 side. For convenience of explanation, illustration of the distal end portion is omitted in FIG. 6.

The jaw 11 is made of a material having conductivity. This jaw 11 is a member in which the jaw main body 111 and a pair of bearing portions 112 are integrated as illustrated in FIG. 6.

The jaw main body 111 is constituted of a long plate-like body. In this jaw main body 111, a concave portion 1111 extending toward the distal end side Ar1 from the proximal end along the longitudinal direction of the jaw main body 111 is arranged on a surface on the treating portion 131 side as illustrated in FIG. 6.

At a substantially central position in the longitudinal direction of the jaw 11 in side wall portions on both sides of a width direction of the jaw main body 111 constituting this concave portion 1111, a through hole 1113 that respectively pierces through a front and a back of the side wall portion is arranged as illustrated in FIG. 6. The third pin Pi3 is fixed to the through hole 1113 by welding in an inserted state.

Moreover, on a surface on a rear side of the jaw main body 111 apart from the treating portion 131, a cover RC (FIG. 2, FIG. 3, FIG. 6) made of an electrically insulating resin is formed in an integrated manner, covering the surface on the rear side. In the first embodiment, the cover RC is formed by insert molding with respect to the jaw main body 111, but it is not limited thereto. For example, a configuration in which the cover RC is fixed to the jaw main body 111 with a snap fit or a metal pin may be adopted.

The pair of bearing portions 112 are arranged on the respective end portions on the proximal end side Ar2 of the jaw main body 111, and are respectively constituted of a plate body facing in the width direction of the jaw main body 111. The pair of bearing portions 112 have the same configuration. Therefore, in the following, only the configuration of one of the bearing portions 112 will be explained.

In the bearing portion 112, first insertion holes 1121 and a second insertion hole 1122 that pierce through a front and a back of the bearing portion 112 are arranged. The bearing portion 112 is connected to the outer pipe 10 when the first pin Pi1 is inserted in the first insertion holes 1121. Moreover, the bearing portion 112 is connected to the inner pipe PI when the second pin Pi2 is inserted in the second insertion hole 1122.

### About Configuration of Holder

FIG. 7 is a diagram explaining a configuration of the holder 14. Specifically, FIG. 7 is a perspective view of the holder 14 as viewed from the treating portion 131 side. In FIG. 7, the electrode EP and the first and the second conductive surfaces CS1 and CS2 are marked with dots for convenience of explanation.

The holder 14 is made of a first material having an electrical insulation property and a biocompatibility. The first material is, for example, a resin, such as polyetheretherketone (PEEK) and polyphenyl sulfone (PPSU), but may be a material having an electrical insulation property, such as ceramics. This holder 14 is a portion in which the holder main body 141, multiple first teeth 142, and multiple second teeth 143 are integrated as illustrated in FIG. 7.

The holder main body 141 is constituted of a long plate body. Moreover, an outer shape of the holder main body 141 is formed to be substantially the same as an inner surface shape of the concave portion 1111.

The first teeth 142 respectively protrude toward the treating portion 131 from one side in the width direction on a surface of the holder main body 141 on the treating portion 131 side, and are aligned along the longitudinal direction of the holder main body 141 as illustrated in FIG. 7.

The second teeth 143 respectively protrude toward the treating portion 131 from the other side in the width direction on a surface of the holder main body 141 on the treating portion 131 side, and are aligned along the longitudinal direction of the holder main body 141 as illustrated in FIG. 7.

As illustrated in FIG. 7, on the surface of the holder main body 141 on the treating portion 131 side, at a central portion positioned between the first teeth 142 and the second teeth 143 in the width direction, a concave portion 144 that recesses toward the side apart from the treating portion 131 and that extends along the longitudinal direction of the holder main body 141 is arranged. Furthermore, at the side wall portions on both sides in the width direction of the holder main body 141 constituting the concave portion 144, claw portions 145 and 146 (FIG. 3, FIG. 7) that protrude toward the center of the width direction and that extend along the longitudinal direction of the holder main body 141 are respectively arranged. The abutting portion 12 is engaged with the claw portions 145 and 146, to thereby be mechanically fixed to the holder 14. That is, the abutting portion 12 is arranged at the central portion of the holder 14 in the width direction. Furthermore, in the side wall portions on both sides of the holder main body 141 in the width direction, through holes 147 that pierce through the front and back of the side wall portions, and in which the third pin Pi3 is inserted are arranged, the holder main body 141 constituting the concave portion 144.

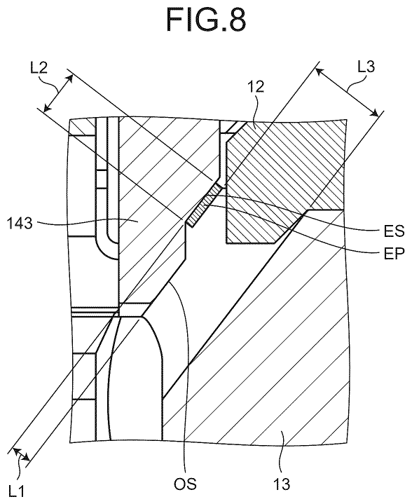

FIG. 8 is a partial enlarged view in which an electrode surface ES in FIG. 3 is enlarged. The second teeth 143 of the holder 14 include an opposing surface OS that faces the ultrasonic blade 13, and the electrode surface ES that is positioned in a direction away from the ultrasonic blade 13 with respect to the opposing surface OS and on which the electrode EP supplying the high frequency current is arranged.

Similarly, the first teeth 142 of the holder 14 include an opposing surface OS that faces the ultrasonic blade 13, and the electrode surface ES that is positioned in a direction away from the ultrasonic blade 13 with respect to the opposing surface OS and on which the electrode EP supplying the high frequency current is arranged. That is, the opposing surface OS and the electrode surface ES are arranged on each of both sides of the abutting portion 12 in the width direction.

A length L1 is, for example, 0.1 mm, but it may be equal to or more than 0.05 mm and equal to or less than 0.3 mm. That is, the electrode surface ES is arranged at a position of 0.05 mm or more and 0.3 mm or less from the opposing surface OS in the direction away from the ultrasonic blade 13.

A length L2 is, for example, 0.33 mm. That is, the width of the electrode EP is 0.33 mm.

A length L3 may be, for example, equal to or more than 0.07 mm. That is, the clearance between the ultrasonic blade 13 and the electrode EP is preferable to be 0.07 mm or more.

### About Configuration of Electrode and First and Second Conductive Surfaces

The electrode EP is arranged at each of the electrode surface ES on an inner side in the width direction of the first teeth 142 (on the second tooth 143 side), the electrode surface ES on the inner side in the width direction of the teeth 143 (on the first tooth 142 side), and the distal end side of the holder 14 to connect these electrode surfaces ES. By arranging the electrode EP also at the distal end portion of the holder 14 also, it becomes possible to treat the treatment target at the distal end also. However, the electrode EP is not necessarily required to be connected at the distal end portion of the holder 14. Moreover, on the electrode EP, a coating material that has non-adhesivity to the treatment target is applied. The coating material is an ultra-thin layer of several hundred nm to several μm in thickness that includes fluorine or silicon.

The first conductive surface CS1 is arranged on the inner surface of the insertion hole 147 as illustrated in FIG. 7.

The second conductive surface CS2 is arranged on the side surface portion of the concave portion 144, and electrically connects the electrode EP and the first conductive surface CS1 as illustrated in FIG. 7.

The electrode EP and the first and the second conductive surfaces CS1 and CS2 explained above are formed by three-dimensional plating processing described below. Specifically, to the holder 14, laser irradiation is applied to a predetermined position. Thereafter, the electrode EP and the first and second conductive surfaces CS1 and CS2 are formed by electroless plating at the laser irradiation position. The thickness of electrode EP and the first and the second conductive surfaces CS1 and CS2 is, for example, around several micrometers. Moreover, a structure fabricated by such three-dimensional plating processing is referred to as a molded interconnect device (MID) in which an electrode circuit is formed on a surface of a three-dimensional resin-molded product. As the electrode EP and the first and the second conductive surfaces CS1 and CS2, not limited to those formed by the three-dimensional plating processing described above, ones formed by other methods maybe adopted.

To apply the high frequency energy to the treatment target, when the high frequency power is supplied to the electrode EP, the high-frequency power is supplied along an electrical path through the electric cable C, the outer pipe 10, the first pin Pi1, the jaw 11, the third pin Pi3, the first conductive surface CS1, the second conductive surface CS2, and the electrode EP.

According to the first embodiment explained above, following effects are produced.

In the treatment tool 2 according to the first embodiment, because the electrode EP is arranged on the electrode surface ES positioned in the direction away from the ultrasonic blade 13 with respect to the opposing surface OS, even when the abutting portion 12 is worn by ultrasonic vibrations, it is possible to suppress generation of sparks caused when the ultrasonic blade 13 and the electrode EP come into contact.

Moreover, the electrode surface ES is a surface that is arranged at a position of 0.05 mm or more and 0.3 mm or less from the opposing surface OS in the direction away from the ultrasonic blade 13. As a result, it is possible to suppress generation of sparks when the ultrasonic blade 13 and the electrode EP come into contact, and to treat the treatment target preferably.

Moreover, the electrode EP is formed by three-dimensional plating processing. Therefore, even when the holder 14 is made of an electrically insulating material, the electrode EP can be formed easily at a specific position in the holder 14.

Furthermore, on the electrode EP, a coating material that has non-adhesivity to a treatment target is provided. Therefore, it is possible to avoid the treatment target from sticking to the electrode EP, and to treat the treatment target preferably.

### Second Embodiment

### Schematic Configuration of Treatment System

An entire configuration of a treatment system 1 according to a second embodiment maybe the same as that of FIG. 1, and explanation thereof is omitted. The same reference symbols will be used to explain configurations similar to those in the treatment system 1, and new reference symbols will be assigned to explain components that differ from those in the treatment system 1.

### Configuration of Treatment Tool

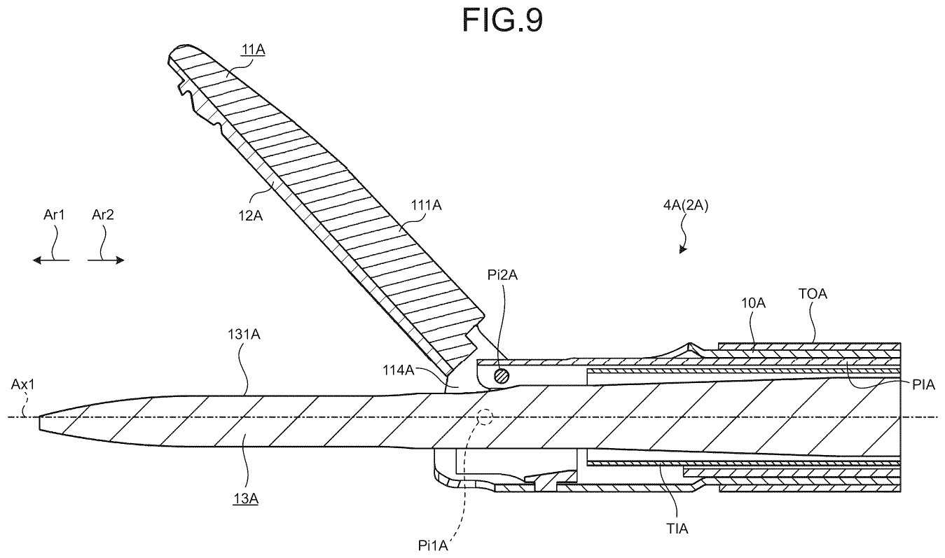

FIG. 9 is a diagram illustrating an ultrasonic treatment tool according to the second embodiment. A handpiece 4A of a treatment tool 2A includes an outer pipe 10A, a jaw 11A, an abutting portion 12A, and an ultrasonic blade 13A as illustrated in FIG. 9.

The outer pipe 10A has a tubular shape and corresponds to a pipe. In the second embodiment, the outer pipe 10A is a pipe in a cylindrical shape that is made of an electrically conductive material, such as metal.

In this outer pipe 10A, a first pin Pi1A (FIG. 1, FIG. 9) that has a cylindrical shape extending in the direction perpendicular to the sheet plane of FIG. 1 and FIG. 9, and that engages with the jaw 11A and axially supports the jaw 11 in a rotatable manner is fixed. In the second embodiment, the first pin Pi1A is made of an electrically conductive material, such as metal. The first pin Pi1A corresponds to a pin.

An outer circumferential surface of the outer pipe 10A is covered with an electrically insulating outer tube TOA (FIG. 9). Moreover, inside the outer pipe 10A, a tubular-shaped inner pipe PIA (FIG. 9) that moves back and forth along a longitudinal direction of the outer pipe 10A in response to the opening closing operation to the operation handle 7 by operator, such as a surgeon, is inserted. At an end portion on the distal end side Ar1 of the inner pipe PIA, a second pin Pi2A (FIG. 9) that has a cylindrical shape extending in the direction perpendicular to the sheet plane of FIG. 1 and FIG. 9, and that engages with the jaw 11A is fixed. In the second embodiment, the second pin Pi2A is arranged on an upper side with respect to the first pin Pi1A in FIG. 9 (a side on which a jaw main body 111A is arranged with respect to a treating portion 131A).

The jaw 11A is connected to the outer pipe 10A by the first pin Pi1A being inserted into an insertion hole of a bearing portion 114A. Moreover, the jaw 11A is connected to the inner pipe PIA by the second pin Pi2A being inserted into an insertion hole of a bearing portion 114A. The jaw 11A pivots around the first pin Pi1A with respect to the outer pipe 10A in conjunction with the back and forth movement of the inner pipe PIA according to the opening closing operation to the operation handle 7 by the operator, such as surgeon. Thus, the jaw 11A opens and closes with respect to the treating portion 131A, which is the end portion on the distal end side in the ultrasonic blade 13, and is enabled to hold the treatment target between itself and the treating portion 131A.

The treatment instrument 2A may be configured as a push-to-close type, or a pull-to-close type. Moreover, as the treatment tool 2A, similarly to the first embodiment, the jaw 11A may be configured to open and close when the inner pipe PIA moves back and forth, or may be configured to open and close when the outer pipe 10A moves back and forth.

The jaw 11A is made of a first material having an electrical insulation property and a biocompatibility. The first material is, for example, a resin, such as polyetheretherketone (PEEK) and polyphenyl sulfone (PPSU), but may be a material having an electrical insulation property, such as ceramics.

The abutting portion 12A is made of a first resin material having an electrical insulation property and a biocompatibility. The first resin material is, for example, polytetrafluoroethylene (PTFE). The abutting portion 12 has a substantially cuboid shape that extends along the longitudinal direction of the jaw 11A. The abutting portion 12A is fixed to a surface on a side closer to the treating portion 131A in a jaw main body 111A, and abuts on the treating portion 131A when the jaw 11A is closed with respect to the treating portion 131A. This abutting portion 12A has a function of preventing breakage caused when the treating portion 131A that is making ultrasonic vibrations collides with the jaw 11A after incision of the treatment target utilizing ultrasonic vibrations is completed.

The ultrasonic blade 13A respectively supplies ultrasonic vibrations and high-frequency current individually to a living tissue. The ultrasonic blade 13A is made of an electrically conductive material, and has a long shape extending along the center axis Ax1. Moreover, the ultrasonic blade 13A is inserted in the inner pipe PIA in a state in which the treating portion 131A protrudes outside as illustrated in FIG. 9. At this time, the end portion on the proximal end side Ar2 of the ultrasonic blade 13A is mechanically connected to an ultrasonic vibrator 52 constituting the ultrasonic transducer 5 as illustrated in FIG. 1. The ultrasonic blade 13A transmits ultrasonic vibrations generated by the ultrasonic transducer 5 from the end portion on the proximal end side Ar2 to the treating portion 131A. The ultrasonic vibrations are vertical vibrations vibrating in the direction along the center axis Ax1. Furthermore, in the ultrasonic blade 13, an outer circumferential surface except the treating portion 131A is covered with the electrically insulating inner tube TIA (FIG. 9).

### About Configuration of Holder

FIG. 10 is a partial enlarged view in which the electrode surface ESA is enlarged. The jaw 11A has multiple second teeth 113A that protrude toward the treating portion 131A from one side in the width direction on the surface on the treating portion 131A side of the jaw main body, and that are aligned along the longitudinal direction of the jaw main body. The second teeth 113A include an opposing surface OSA that faces the ultrasonic blade 13A, and the electrode surface ESA that is positioned in a direction away from the ultrasonic blade 13A with respect to the opposing surface OSA and on which the electrode EPA supplying the high frequency current is arranged.

A length LIA is, for example, 0.1 mm, but it may be equal to or more than 0.05 mm and equal to or less than 0.3 mm. That is, the electrode surface ESA is a surface that is arranged at a position of 0.05 mm or more and 0.3 mm or less from the opposing surface OSA in the direction away from the ultrasonic blade 13A.

A length L2A is, for example, 0.33 mm. That is, the width of the electrode EPA is 0.33 mm.

A length L3A may be, for example, equal to or more than 0.07 mm. That is, the clearance between the ultrasonic blade 13A and the electrode EPA is preferable to be 0.07 mm or more.

Similarly, the jaw 11A has multiple first teeth that protrude toward the treating portion 131A from the other side in the width direction on the surface on the treating portion 131A side of the jaw main body, and that are aligned along the longitudinal direction of the jaw main body. The first teeth include an opposing surface OSA that faces the ultrasonic blade 13A, and the electrode surface ESA that is positioned in a direction away from the ultrasonic blade 13A with respect to the opposing surface OSA and on which the electrode EPA supplying the high frequency current is arranged. That is, the opposing surface OSA and the electrode surface ESA are arranged on each of both sides of the abutting portion 12A in the width direction.

According to the second embodiment explained above, following effects are produced.

In the treatment tool 2A according to the second embodiment, because the electrode EPA is arranged on the electrode surface ESA positioned in the direction away from the ultrasonic blade 13A with respect to the opposing surface OSA, even when the abutting portion 12A is worn by ultrasonic vibrations, it is possible to suppress generation of sparks caused when the ultrasonic blade 13A and the electrode EPA come into contact.

Moreover, the electrode surface ESA is a surface that is positioned in the direction away from the ultrasonic blade 13A with respect to the opposing surface OSA by 0.05 mm or more and 0.3 mm or less. As a result, it is possible to suppress generation of sparks when the ultrasonic blade 13A and the electrode EPA come into contact, and to treat the treatment target preferably.

Moreover, the electrode EPA is formed by three-dimensional plating processing. Therefore, even when the jaw 11A is made of an electrically insulating material, the electrode EPA can be formed easily at a specific position in the jaw 11A.

Moreover, on the electrode EPA, a coating material that has non-adhesivity to a treatment target is provided. Therefore, it is possible to avoid the treatment target from sticking to the electrode EPA, and to treat the treatment target preferably.

### Third Embodiment

In the first embodiment and the second embodiment, the treatment tools 2, 2A adopt the ultrasonic energy and the high frequency energy as the treatment energy to be applied to a treatment target, but it is not limited thereto, and only the high frequency energy may be used.

A treatment tool according to a third embodiment includes a pair of jaws that open and close to hold a cutter from top and bottom. The jaw is a portion that holds a treatment target, and that treats the treatment target by applying the high frequency energy to the treatment target.

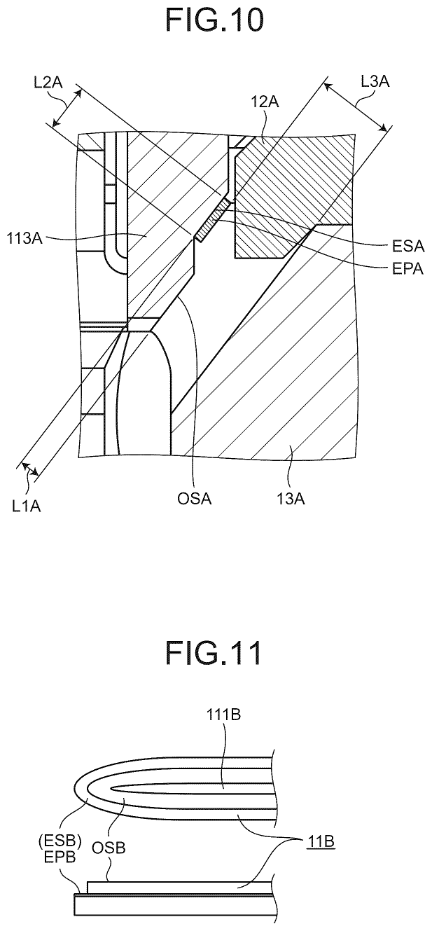

FIG. 11 is a diagram illustrating an upper surface and a side surface of the jaw of the treatment tool according to the third embodiment. FIG. 11 illustrates an upper surface and a side surface of a jaw 11B positioned below the cutter. The jaw 11B includes an opposing surface OSB facing the cutter, an electrode surface ESB that is positioned in a direction away from the cutter with respect to the opposing surface OSB and on which an electrode EPB supplying the high frequency current is arranged, and a cutter groove 111B that extends toward the longitudinal direction of the jaw 11B.

The jaw 11B is made of a first material having an electrical insulation property and a biocompatibility. The first material is, for example, a resin, such as polyetheretherketone (PEEK) and polyphenyl sulfone (PPSU), but may be a material having an electrical insulation property, such as ceramics.

The electrode EPB is a portion to which the high frequency current is supplied from the control device 3 between the opposing electrodes EPB. This electrode EPB has a U-shape surrounding the cutter groove 111B in a planar manner, and is arranged on a surface of the jaw 11B on the cutter side in such a position that both ends of the U-shape are directed toward the proximal end side Ar2.

The electrode EPB is formed by three-dimensional plating processing, similarly to the electrode EP explained in the first embodiment. As the electrode, not limited to those formed by the three-dimensional plating processing described above, ones formed by other methods maybe adopted.

Furthermore, on the electrode EPB, a coating material that has non-adhesivity to a treatment target is provided. The coating material is an ultra-thin layer of several hundred nm to several μm in thickness that includes fluorine or silicon.

According to the third embodiment explained above, following effects are produced.

In the treatment tool according to the third embodiment, because the electrode EPB is arranged on the electrode surface ESB positioned in the direction away from the cutter with respect to the opposing surface OSB, it is possible to prevent contact between the cutter and the electrode EPB.

Moreover, the electrode EPB is formed by three-dimensional plating processing. Therefore, even when the jaw 11B is made of an electrically insulating material, the electrode EPB can be formed easily at a specific position in the jaw 11B.

Furthermore, on the electrode EPB, a coating material that has non-adhesivity to a treatment target is provided. Therefore, it is possible to avoid the treatment target from sticking to the electrode EPB, and to treat the treatment target preferably.

According to the disclosure, an ultrasonic treatment tool that is capable of suppressing generation of sparks can be realized.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment tool comprising:
an ultrasonic blade configured to supply ultrasonic vibration and high frequency current to a living tissue;
a jaw configured to open and close with respect to the ultrasonic blade;
a holder that is supported by the jaw, and that is made of a first material having an electrical insulation property;
an abutting portion that is arranged in the holder, and that is made of a first resin material, the abutting portion being configured to abut on the ultrasonic blade when the jaw is closed with respect to the ultrasonic blade; and
a first pin that is provided in the jaw to support the holder in a swingable manner,
the holder including
an opposing surface that faces the ultrasonic blade, and
an electrode surface that is positioned in a direction away from the ultrasonic blade with respect to the opposing surface and on which an electrode is arranged, the electrode being electrically connected to the jaw through the first pin to supply the high frequency current.

2. The ultrasonic treatment tool according to the first embodiment, wherein
the abutting portion is arranged at a central portion of the holder in a width direction, and
the opposing surface and the electrode surface are arranged on each of both sides of the abutting portion in the width direction.

3. The ultrasonic treatment tool according to claim 1, wherein
the first material is resin.

4. The ultrasonic treatment tool according to claim 3, wherein
the first material is polyetheretherketone or polyphenyl sulfone.

5. The ultrasonic treatment tool according to claim 1, wherein
the electrode is formed by three-dimensional plating processing.

6. The ultrasonic treatment tool according to claim 1, wherein
the first pin has a cylindrical shape.

7. The ultrasonic treatment tool according to claim 6, wherein
the holder includes an insertion hole in which the first pin is inserted, and
an inner surface of the insertion hole includes a first conductive surface that electrically connects the first pin and the electrode.

8. The ultrasonic treatment tool according to claim 7, wherein
the holder includes a second conductive surface that electrically connects the first conductive surface and the electrode.

9. The ultrasonic treatment tool according to claim 1, wherein
the holder is swingable with respect to the jaw.

10. The ultrasonic treatment tool according to claim 1, further comprising:
a second pin in a cylindrical shape; and
a pipe to which the second pin is attached to rotatably support the jaw with the second pin.

11. The ultrasonic treatment tool according to claim 1, wherein
the electrode surface is arranged at a position of 0.05 mm or more and 0.3 mm or less from the opposing surface in the direction away from the ultrasonic blade.

12. The ultrasonic treatment tool according to claim 1, wherein
a coating material having non-adhesivity to the living tissue is arranged on the electrode.
